(19) United States
(12) Patent Application Publication
(10) Pub. No.: US 2009/0252674 A1
(43) Pub. Date: Oct. 8, 2009

(54) Tripodal Ligands with the Coordinating Motifs K2 -BH2 or K3 -BH3 Relevant for Biomedical Applications of Organometallic Complexes

(75) Inventors: Isabel Rego Santos, Lisboa (PT); Antonio Manuel Rocha Paulo, Lisboa (PT)

Correspondence Address: Mallinckrodt Inc., 675 McDonnell Boulevard, HAZELWOOD, MO, 63042, US

(73) Assignee: Mallinckrodt Inc., Hazelwood, MO (US)

(21) Appl. No.: 12/097,553

(22) PCT Filed: Dec. 15, 2006
(86) PCT No.: PCT/US06/47877

§ 371 Date: Jun. 16, 2008

(30) Foreign Application Priority Data
Dec. 23, 2005 (EP) 05077977.6
Jan. 19, 2006 (EP) 06075127.8

(51) Int. Cl. A61K 51/04; C07F 13/00; A61K 103/10; A61K 103/00; A61P 35/00

(52) U.S. Cl. 424/1.65; 534/14; 534/10

(57) ABSTRACT

The present invention relates to a compound for use as a chelator in the radioactive labeling of biomolecules with metal tricarbonyl complexes, which compound has the general Formula (I) wherein M is a monovalent cation, such as Li, Na, K, Tl, Rb, Cs or an alkyl ammonium; R1 is H, alkyl, aryl or a biomolecule; R2 is H or a pendant arm, said pendant arm optionally comprises a biomolecule, with the proviso that when R1 is H, R2 is not H or COOH, and when R1 is alkyl or aryl, R2 is not H.

The present invention relates to new tripodal di- and trihydroborate ligands for use in radioactive labeling of, for example, biomolecules. The invention further relates to methods for labeling with these new ligands and to compounds labeled by means of these ligands. Furthermore, the invention relates to the use of such labeled compounds in diagnosis and therapy.

Tc(I) and Re(I) tricarbonyl complexes have potential relevance in the development of radioactive products for diagnostic (^{99m}Tc) or therapeutic (^{186/188}Re) medical applications. The use of fac−[M(CO)_{3]}⁺ (M=Re, Tc) moieties for labeling biomolecules implies, however, the use of bifunctional chelators. This is a tremendous challenge as the incorporation of a d-transition metal into a bioactive molecule needs to be done without affecting the metabolical fate and/or binding affinity of the biomolecule.

For biomolecules of relatively low molecular weight, such as CNS or estrogen receptor ligands, amino acids and enzyme substrates or inhibitors, this challenge is even higher as these small biomolecules, after coordination to the metal fragment, should still be able to recognize the corresponding receptors, enzymes and/or transporters in a specific way. So, the metal fragments must be as small as possible, and must have physico-chemical properties compatible with the biomolecule.

The present invention is directed to novel ligands that are useful for labeling different types of biomolecules with metal tricarbonyl complexes.

In the present invention tripodal di- and trihydroborate ligands were developed, which may stabilize metal tricarbonyl complexes through two hydrogen atoms and a third coordinating arm or through three hydrogen atoms. The neutral organometallic compounds of formula II, containing κ²-BH₂ or κ³-BH₃ motifs, can be applied as building blocks for the labeling of a variety of biomolecules, in particular biomolecules of a low molecular weight with potential interest for radiopharmaceutical application.

The present invention thus relates to tripodal chelators of the general formula I

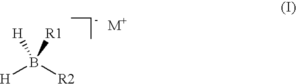

wherein:
M is a monovalent cation, such as, for example, Li, Na, K, Tl, Rb, Cs or an alkyl ammonium;
R1 is H, alkyl, aryl or a biomolecule;
R2 is H or a pendant arm, which arm optionally is or comprises a biomolecule,
with the proviso that when R1 is H, R2 is not H or COOH, and when R1 is alkyl or aryl, R2 is not H.

M is preferably Li, Na or K, more preferably Li or Na.

The pendant arm may be a coordinating or non-coordinating arm. The pendant arm preferably comprises, especially when R1 is other than H, at least one heteroatom, such as N, S or O. In preferred embodiments, the pendant arm comprises at least one heteroatom, regardless of whether it is coordinating or non-coordinating.

The alkyl groups can be cyclic or acyclic, linear or branched, saturated or unsaturated. The cyclic and acyclic alkyl groups are optionally substituted with, for example, alkyl groups such as, for example, methyl, ethyl, n-propyl, or isopropyl, or with functional groups such as carboxyl (—COOH), ester (—COOR, wherein R is alkyl or aryl), amines (—NHR, wherein R is H, alkyl or aryl), aldehydes (—(C═O)H), hydroxy (—OH), or mercapto (—SH). In a specific embodiment, alkyl is a C₁-C₆ alkyl, in particular an alkyl selected from methyl, ethyl, n-propyl, isopropyl, n-butyl, isobutyl, s-butyl, t-butyl, n-pentyl, isopentyl, neopentyl, hexyl, 2-methylpentyl, 3-methylpentyl, 2,3-dimethylbutyl and 2,2-dimethylbutyl. In certain embodiments, R1 is alkyl selected from methyl, ethyl, n-propyl and isopropyl. Methyl is particularly preferred when R1 is alkyl.

The aryl groups can be monocyclic (containing 5-8 atoms in the ring structure) or polycyclic (containing 9-18 atoms in the ring structure), and may or may not have alkyl substituents, such as, for example, methyl, ethyl, n-propyl, or isopropyl. The polycyclic aryls can have one or more rings that are not aromatic.

Optionally the aryl groups and/or alkyl substituents attached thereto, or the alkyl group (in the event that R1 is alkyl), can be substituted with functional groups such as carboxyl (—COOH), ester (—COOR, wherein R is alkyl or aryl), amines (—NHR, wherein R is H, alkyl or aryl), aldehydes (—(C═O)H), hydroxy (—OH), or mercapto (—SH) for coupling biomolecules. In certain embodiments, a biomolecule is coupled to the alkyl or aryl of R1.

In a specific embodiment, the aryl, which is preferably phenyl, is optionally substituted with one or more alkyl groups selected from methyl, ethyl, n-propyl, n-butyl, isobutyl, s-butyl, t-butyl, n-pentyl, isopentyl, neopentyl, hexyl, 2-methylpentyl, 3-methylpentyl, 2,3-dimethylbutyl, and 2,2-dimethylbutyl.

When the aryl is a heterocyclic aromatic group it contains one or more heteroatoms, such as N, S, and/or O, (e.g. mercaptoimidazole, mercapto-oxazole, mercaptothiazole, pyrrole, furan, thiophene, imidazole, oxazole, thiazole, pyrazole, pyridine, and pyrimidine). Cyclic coordinating or non-coordinating arms may contain one or more heteroatoms, such as N, S, and/or O, and are, for example, piperidine, thiazoline, oxazoline, and the like. These groups may or may not comprise a biomolecule.

A biomolecule as used herein is a chemical compound/substituent that naturally occurs in living organisms or, if it does not occur naturally in living organisms, is a chemical compound/substituent that can recognize other biologically active molecules existing in living organisms. Biomolecules consist primarily of carbon and hydrogen, along with nitrogen, oxygen, phosphorus and/or sulfur. Other elements, such as Fe(2+), Fe(3+), Co(3+) or Zn(2+), sometimes are incorporated but these are much less common.

When R2 is a coordinating or non-coordinating pendant arm, it can be acyclic (linear or branched), cyclic (i.e. containing a ring of atoms in the nucleus, which ring is formed by several atoms linked together, and which ring may be monocyclic or polycyclic (having two or more rings), the rings being saturated, unsaturated, aromatic, or mixtures thereof (in the case of polycyclic structures)).

When R2 is a non-coordinating arm, R1 has to be H. R2 can be non-coordinating in case it does not comprise a coordinating atom, e.g., such as a heteroatom, or group or in case the coordinating atom or group is too far removed from the boron atom to coordinate to the metal. In case R1 is coordinating, R2 need not be coordinating and can be a biomolecule.

A coordinating arm, as used herein, is a group that is capable of binding to a metal ion by an ionic or covalent bond.

Examples of coordinating arms that are suitable for use in this invention are mercaptoimidazoles, mercaptothiazoles, mercaptobenzothiazoles, mercaptobenzimidazoles, mercaptobenzoxazoles, mercapto-oxazoles, mercaptoimidazolines, mercaptothiazolines, mercapto-oxazolines, or groups containing thioethers, carboxylates or amines. In particular embodiments, coordinating pendant arms include mercaptoimidazoles, mercaptothiazolines and mercaptobenzothiazoles.

The coordinating arms may be optionally substituted with alkyls, aryls, or functional groups for coupling biomolecules, such as, for example, carboxyl (—COOH), amine (—NHR or NH₂, wherein R is alkyl or aryl as defined above), aldehyde (—(C═O)H), hydroxy (—OH), mercapto (—SH) or ester (—COOR, wherein R is alkyl or aryl as defined above) groups. Selection of suitable coordinating arms leads to a strong chelation and enables introduction of different biomolecules in different positions of these groups.

The substituents introduced in these coordinating groups can be linear, cyclic or polycyclic. Examples of suitable substituents include alkyls selected from methyl, ethyl, n-propyl and isopropyl, and aryls such as phenyl that is optionally substituted with alkyls selected from methyl, ethyl, n-propyl or isopropyl.

When R2 is a non-coordinating group, R2 can be a biomolecule or an alkyl or aryl, as defined above, containing a functional group, such as a carboxyl (—COOH), an amine (—NHR or —NH₂, wherein R is an alkyl or aryl as defined above), aldehyde (—(C═O)H), hydroxy (—OH), mercapto (—SH) or ester (—COOR, wherein R is allyl or aryl as defined above) group for coupling biomolecules.

Compounds that are excluded from the above definition for formula I by means of the proviso are not claimed per se but are still suitable for the use aspects of the invention, which is for radioactive labeling of biomolecules. This use is claimed for all compounds that fall within the definition of formula I without the proviso.

The compounds of the invention allow the synthesis of tricarbonyl metal fragments with the coordinating motifs κ²-H₂B or κ³-H₃B.

The present invention is thus useful for labeling biomolecules in general. In one embodiment, particularly suitable biomolecules are those of low molecular weight, e.g. below 1000 Da. The following is a non-limiting list of examples: CNS receptor ligands, steroids, sugars, amino acids, substrates or inhibitors of enzymes, or other molecules with affinity for receptors over-expressed in certain tumor cells. Examples of CNS receptor ligands are antagonists of dopamine and serotonin transporters, dopamine and serotonin receptor ligands or molecules for the targeting of β-amyloid plaques, such as thioflavine or chrysamine derivatives. The invention is useful for labeling steroids such as estrogens, progestens and androgens and derivatives thereof, or sugars such as glucose or mannose or derivatives thereof. The invention is also suitable for radioactive labeling of molecules with affinity for transporters or receptors over-expressed in certain tumor cells, such as L-type amino acid transporters, peptide receptors (e.g., somatostatin, bombesin or CCK-gastrin receptors), and sigma receptors.

The invention relates to various types of compounds that optionally comprise a biomolecule. If a biomolecule is present it can be in the position of R1 or R2 or be attached to R1 or R2. In a further embodiment both R1 and the substituent on R2 are biomolecules. Moreover, R2 can be a biomolecule when R1 is H or any other coordinating group, which coordinating group optionally contains a biomolecule or a functional group to couple a biomolecule.

According to a further aspect thereof, the invention relates to a method for the preparation of a compound of formula I as described above, comprising reacting a compound of the general formula H₃BR1, wherein R1 is H, alkyl, aryl or a biomolecule as defined above, with a compound selected from mercaptoimidazoles, mercaptobenzothiazoles, mercaptothiazoles, mercapto-oxazoles, mercaptoimidazolines, mercaptothiazolines, mercapto-oxazolines or any one of these compounds linked to a biomolecule.

The invention further relates to compounds, i.e. organometallic complexes, of the general formula II:

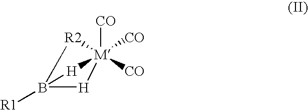

wherein M′ is a transition metal, in particular a group VII metal, and R1 and R2 are as defined above. In one embodiment, M′ is selected from rhenium, technetium or manganese, more particularly a radioactive isotope of rhenium, technetium or manganese, and in particular ^{99m}Tc or ^{186/188}Re.

In one particular embodiment, R1 is a biomolecule. In another embodiment, R2 is a coordinating arm containing a biomolecule. In a further embodiment, R1 is a biomolecule and R2 is a coordinating arm containing a biomolecule.

According to a further aspect thereof, the invention relates to a method for the preparation of a compound of formula II, comprising contacting a metal tricarbonyl with a compound of formula I.

Labeling of biomolecules can be achieved by starting from a compound of formula I that is already substituted with a biomolecule and coordinating that compound to the radioactive metal tricarbonyl. As an alternative, a compound according to formula II can be prepared in which no biomolecule is present yet. A biomolecule can then be linked to R2 or R1 or to both provided that R1 and/or R2 contain functional groups, such as, for example, amines (—NHR, wherein R is H, alkyl or aryl), aldehydes (—(C═O)H), carboxyl (—COOH), hydroxy (—OH), mercapto (—SH), or ester (—COOR, wherein R is alkyl or aryl) groups, which can react with appropriate functional groups existing in the biomolecule.

The transition metal tricarbonyl complexes that can be prepared with the compounds of formula I of the invention can be used in nuclear medicine. Depending on the specific R1 and R2 of formula I, the metal tricarbonyl complexes may be used, for example, for diagnosis and therapy of cancer or other hyperproliferative and/or neoplasic conditions, or for diagnosis of central nervous system diseases. A preferred use of the complexes is in diagnosis and/or therapy of cancer.

The invention thus also relates to the use of a compound of formula II having a suitable biomolecule at, or linked to, R1 and/or a suitable biomolecule at, or linked to, R2 for the preparation of a pharmaceutical composition for the treatment or diagnosis of cancer or other hyperproliferative and/or neoplasic conditions.

Examples of the compounds of the present invention are listed in table 1 below which shows representative combinations of R1 and R2.

TABLE 1

| Compound | R1 | R2 |
|---|---|---|
| 1 | H | groups |
| | | containing a |
| | | thioether |
| 2 | H | mercapto- |
| | | imidazoles |
| 3 | H | mercapto- |
| | | thiazoles |
| 4 | H | mercapto- |
| | | oxazoles |
| 5 | H | mercapto- |
| | | imidazolines |
| 6 | H | mercapto- |
| | | thiazolines |
| 7 | H | mercapto- |
| | | oxazolines |
| 8 | H | groups |
| | | containing an |
| | | amine |
| 9 | H | groups |
| | | containing a |
| | | carboxylate |
| 10 | methyl | groups |
| | | containing a |
| | | thioether |
| 11 | methyl | mercapto- |
| | | imidazoles |
| 12 | methyl | mercapto- |
| | | thiazoles |
| 13 | methyl | mercapto- |
| | | oxazoles |
| 14 | methyl | mercapto- |
| | | imidazolines |
| 15 | methyl | mercapto- |
| | | thiazolines |
| 16 | methyl | mercapto- |
| | | oxazolines |
| 17 | methyl | groups |
| | | containing an |
| | | amine |
| 18 | methyl | groups |
| | | containing a |
| | | carboxylate |
| 19 | ethyl | groups |
| | | containing a |
| | | thioether |
| 20 | ethyl | mercapto- |
| | | imidazoles |
| 21 | ethyl | mercapto- |
| | | thiazoles |
| 22 | ethyl | mercapto- |
| | | oxazoles |
| 23 | ethyl | mercapto- |
| | | imidaxolines |
| 24 | ethyl | mercapto- |
| | | thiazolines |
| 25 | ethyl | mercapto- |
| | | oxazolines |
| 26 | ethyl | groups |
| | | containing an |
| | | amine |
| 27 | ethyl | groups |
| | | containing a |
| | | carboxylate |
| 28 | propyl | groups |
| | | containing a |
| | | thioether |
| 29 | propyl | mercapto- |
| | | imidazoles |
| 30 | propyl | mercapto-- |
| | | thiazoles |
| 31 | propyl | mercapto- |
| | | oxazoles |
| 32 | propyl | mercapto- |
| | | imidazolines |
| 33 | propyl | mercapto- |
| | | thiazolines |
| 34 | propyl | mercapto- |
| | | oxazolines |
| 35 | propyl | groups |
| | | containing an |
| | | amine |
| 36 | propyl | groups |
| | | containing a |
| | | carboxylate |
| 37 | isopropyl | groups |
| | | containing a |
| | | thioether |
| 38 | isopropyl | mercapto- |
| | | imidazoles |
| 39 | isopropyl | mercapto- |
| | | thiazoles |
| 40 | isopropyl | mercapto- |
| | | oxazoles |
| 41 | isopropyl | mercapto- |
| | | imidazolines |
| 42 | isopropyl | mercapto- |
| | | thiazolines |
| 43 | isopropyl | mercapto- |
| | | oxazolines |
| 44 | isopropyl | groups |
| | | containing an |
| | | amine |
| 45 | isopropyl | groups |
| | | containing a |
| | | carboxylate |
| 46 | phenyl* | groups |
| | | containing a |
| | | thioether |
| 47 | phenyl* | mercapto- |
| | | imidazoles |
| 48 | phenyl* | mercapto- |
| | | thiazoles |
| 49 | phenyl* | mercapto- |
| | | oxazoles |
| 50 | phenyl* | mercapto- |
| | | imidazolines |
| 51 | phenyl* | mercapto- |
| | | thiazolines |
| 52 | phenyl* | mercapto- |
| | | oxazolines |
| 53 | phenyl* | groups |
| | | containing an |
| | | amine |
| 54 | phenyl* | groups |
| | | containing a |
| | | carboxylate |
| 55 | biomolecule | groups |
| | | containing a |
| | | thioether |
| 56 | biomolecule | mercapto- |
| | | imidazoles |
| 57 | biomolecule | mercapto- |
| | | thiazoles |
| 58 | biomolecule | mercapto- |
| | | oxazoles |
| 59 | biomolecule | mercapto- |
| | | imidazolines |
| 60 | biomolecule | mercapto- |
| | | thiazolines |
| 61 | biomolecule | mercapto- |
| | | oxazolines |
| 62 | biomolecule | groups |
| | | containing an |
| | | amine |
| 63 | biomolecule | groups |
| | | containing a |
| | | carboxylate |
| 64 | H | thioether-BM |
| 65 | H | mercapto- |
| | | imidazoles-BM |
| 66 | H | mercapto- |
| | | thiazoles-BM |
| 67 | H | mercapto- |
| | | oxazoles-BM |
| 68 | H | mercapto- |
| | | imidazolines-BM |
| 69 | H | mercapto- |
| | | thiazolines-BM |
| 70 | H | mercapto- |
| | | oxazolines-BM |
| 71 | H | amine-BM |
| 72 | H | carboxylate-BM |
| 73 | methyl | thioether-BM |
| 74 | methyl | mercapto- |
| | | imidazoles-BM |
| 75 | methyl | mercapto- |
| | | thiazoles-BM |
| 76 | methyl | mercapto- |
| | | oxazoles-BM |
| 77 | methyl | mercapto- |
| | | imidazolines-BM |
| 78 | methyl | mercapto- |
| | | thiazolines-BM |
| 79 | methyl | mercapto- |
| | | oxazolines-BM |
| 80 | methyl | amine-BM |
| 81 | methyl | carboxylate-BM |
| 82 | ethyl | thioether-BM |
| 83 | ethyl | mercapto- |
| | | imidazoles-BM |
| 84 | ethyl | mercapto- |
| | | thiazoles-BM |
| 85 | ethyl | mercapto- |
| | | oxazoles-BM |
| 86 | ethyl | mercapto- |
| | | imidazolines-BM |
| 87 | ethyl | mercapto- |
| | | thiazolines-BM |
| 88 | ethyl | mercapto- |
| | | oxazolines-BM |
| 89 | ethyl | amine-BM |
| 90 | ethyl | carboxylate-BM |
| 91 | propyl | thioether-BM |
| 92 | propyl | mercapto- |
| | | imidazoles-BM |
| 93 | propyl | mercapto- |
| | | thiazoles-BM |
| 94 | propyl | mercapto- |
| | | oxazoles-BM |
| 95 | propyl | mercapto- |
| | | imidazolines-BM |
| 96 | propyl | mercapto- |
| | | thiazolines-BM |
| 97 | propyl | mercapto- |
| | | oxazolines-BM |
| 98 | propyl | amine-BM |
| 99 | propyl | carboxylate-BM |
| 100 | isopropyl | thioether-BM |
| 101 | isopropyl | mercapto- |
| | | imidazoles-BM |
| 102 | isopropyl | mercapto- |
| | | thiazoles-BM |
| 103 | isopropyl | mercapto- |
| | | oxazoles-BM |
| 104 | isopropyl | mercapto- |
| | | imidazolines-BM |
| 105 | isopropyl | mercapto- |
| | | thiazolines-BM |
| 106 | isopropyl | mercapto- |
| | | oxazolines-BM |
| 107 | isopropyl | amine-BM |
| 108 | isopropyl | carboxylate-BM |
| 109 | phenyl* | thioether-BM |
| 110 | phenyl* | mercapto- |
| | | imidazoles-BM |
| 111 | phenyl* | mercapto- |
| | | thiazoles-BM |
| 112 | phenyl* | mercapto- |
| | | oxazoles-BM |
| 113 | phenyl* | mercapto- |
| | | imidazolines-BM |
| 114 | phenyl* | mercapto- |
| | | thiazolines-BM |
| 115 | phenyl* | mercapto- |
| | | oxazolines-BM |
| 116 | phenyl* | groups |
| | | containing an |
| | | amine-BM |
| 117 | phenyl* | carboxylate-BM |
| 118 | biomolecule | thioether-BM |
| 119 | biomolecule | mercapto- |
| | | imidazoles-BM |
| 120 | biomolecule | mercapto- |
| | | thiazoles-BM |
| 121 | biomolecule | mercapto- |
| | | oxazoles-BM |
| 122 | biomolecule | mercapto- |
| | | imidazolines-BM |
| 123 | biomolecule | mercapto- |
| | | thiazolines-BM |
| 124 | biomolecule | mercapto- |
| | | oxazolines-BM |
| 125 | biomolecule | amine-BM |
| 126 | biomolecule | carboxylate-BM |
| 127 | H | biomolecule |

*“phenyl” means “phenyl” or “phenyl substituted with methyl, ethyl, n-propyl, isopropyl, n-butyl, isobutyl, s-butyl, t-butyl, n-pentyl, isopentyl, neopentyl, hexyl, 2-methylpentyl, 3-methylpentyl, 2,3-dimethylbutyl, or 2,2-dimethylbutyl”
BM = biomolecule

The presence of the coordinating κ²-BH₂ or κ³-BH₃ motifs in the compounds of the invention, involving hydrogen atoms, leads to metal fragments that are expected to label biomolecules without affecting the metabolic fate and/or binding affinity for specific receptors, transporters or enzymes whose density is altered in diseased tissues.

The compounds of the invention are tridentate bifunctional di- and trihydroborate ligands. The coordinating or non-coordinating pendant arms may contain a functional group to couple biomolecules or may incorporate biomolecules. The functional groups to couple biomolecules, or the biomolecules, can be introduced in different positions of the pendant arm. The alkyl groups can be linear or branched (C₁-C₆) and the aryl groups can be monocyclic or polycyclic as defined above. When substituted they can have carboxyl (—COOH), amine (NHR, wherein R is H, alkyl or aryl as defined above), aldehyde (—C(═O)H), hydroxy (—OH), mercapto (—SH) or ester (—COOR, wherein R is alkyl or aryl as defined above) groups.

Suitable biomolecules are CNS receptor ligands (as for example, for the targeting of dopamine or serotonin transporters, dopamine or serotonin receptors, or β-amyloid plaques), steroids, sugars, amino acids, substrates or inhibitors of enzymes up-regulated in tumor cells (such as, for example, L-arginine or quinazoline derivatives), or molecules with affinity for receptors overexpressed in certain tumor cells (such as, for example, low molecular weight peptides, such as bombesin, somatostatin, or CCK-gastrin analogues).

Exemplary general synthetic procedures for chelators of formula I and organometallic complexes with some of these chelators are shown in schemes 1-3. The suitable reaction conditions for the synthetic procedures shown in schemes 1-3 would be well known to one of ordinary skill in the art having regard to the present disclosure.

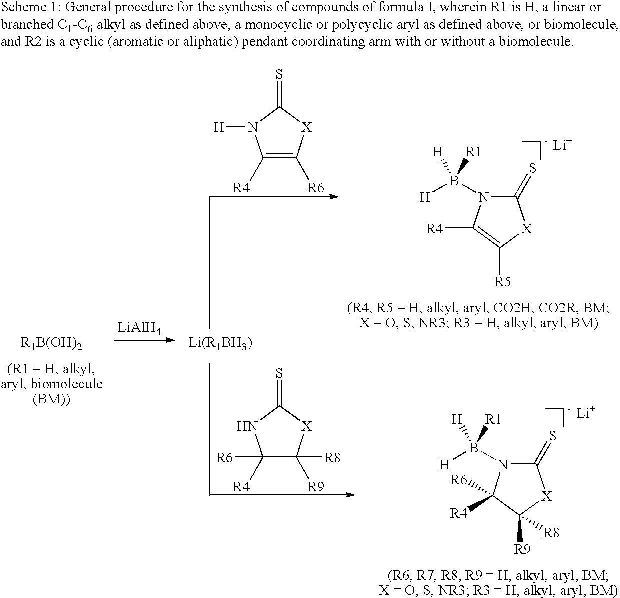

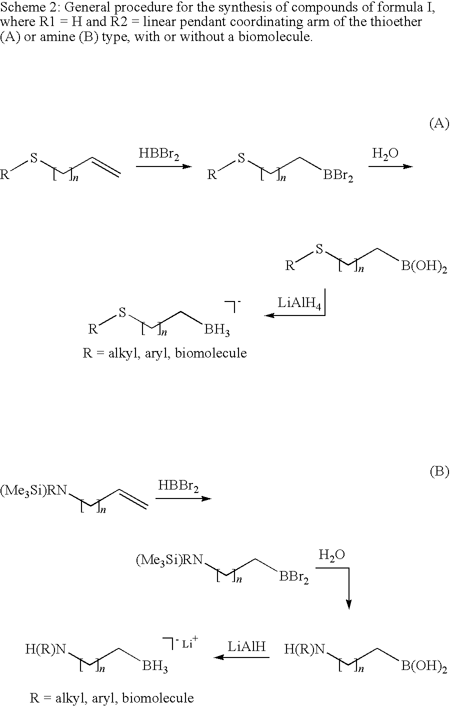

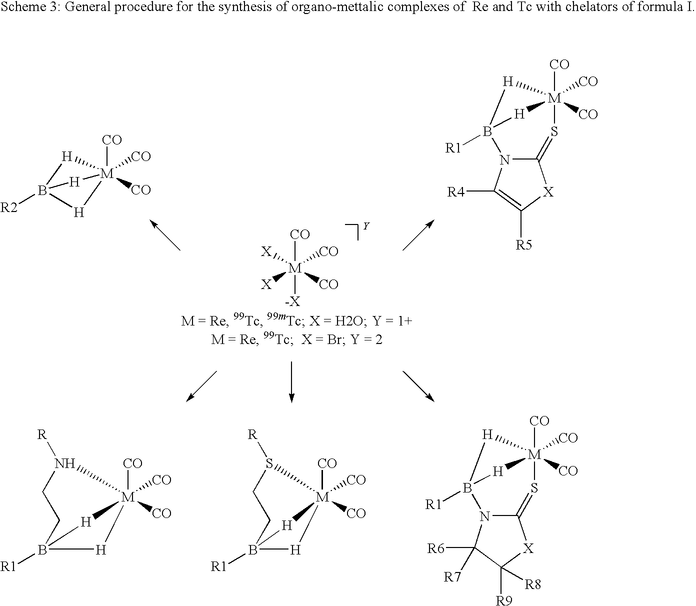

In this application the terms “chelator” or “chelators” and “ligand or “ligands” may be used interchangeably but are intended (unless the context necessitates otherwise) to mean compounds of formula I.

Some examples are given below, to show how to synthesize some of the chelators and how they are suitable to stabilize complexes with the fac−[M(CO)₃]⁺ [M=Re, TC) moiety. These examples solely intend to demonstrate the invention, but are in no way intended to be limiting to the scope thereof.

In the examples, reference is made to the following figures:

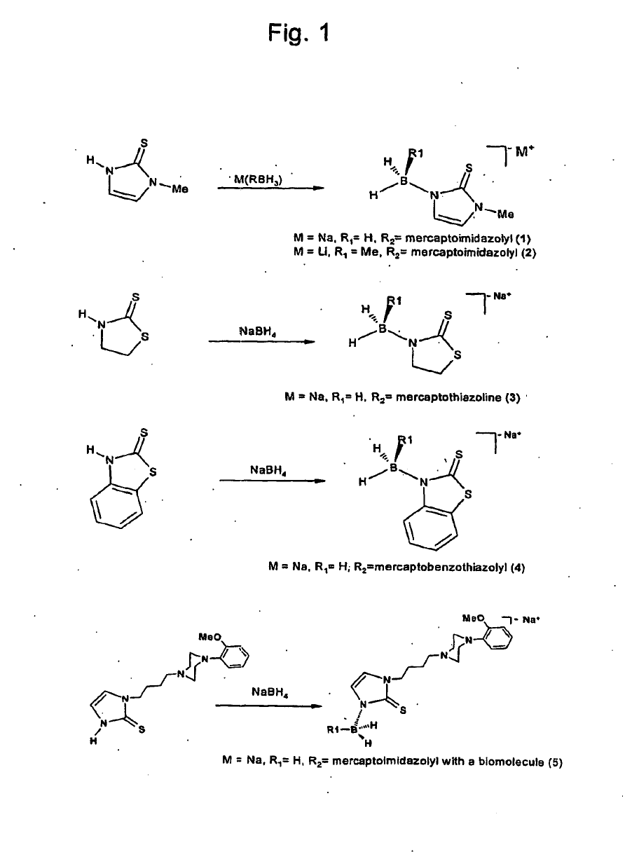

FIG. 1 shows a schematic representation of the synthesis of representative examples of chelators of formula I (specifically compounds 1, 2, 3, 4, 5).

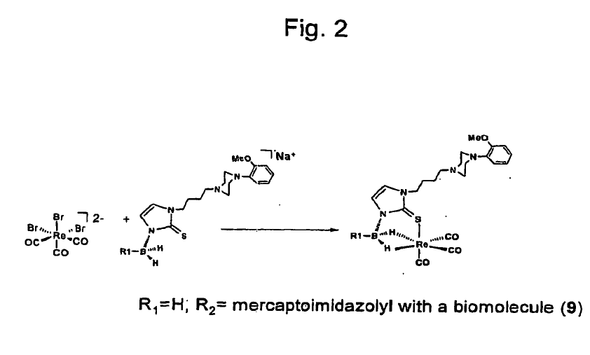

FIG. 2 shows a schematic representation of the synthesis of representative examples of a Re(I) tricarbonyl complex with one of the chelators of formula I (specifically complex 9 isolated with compound 5).

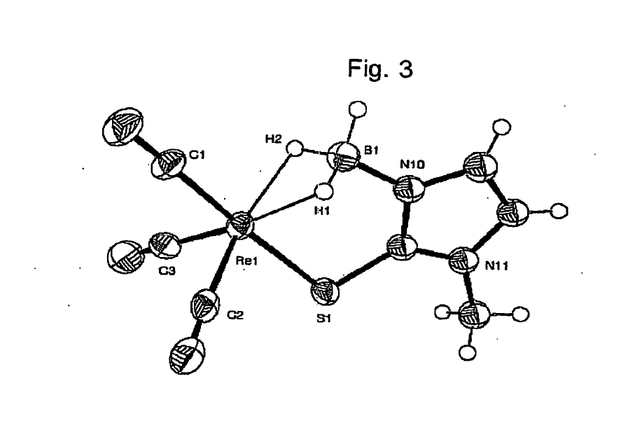

FIG. 3 shows the molecular structure of a Re(I) tricarbonyl complex with one of the chelators of formula I (specifically complex 6 isolated with compound 1).

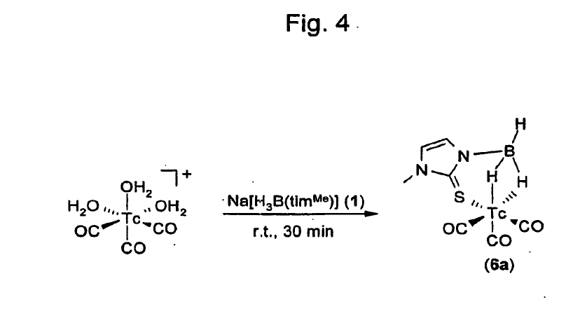

FIG. 4 shows the synthesis of a ^{99m}Tc tricarbonyl complex with one of the chelators of formula I.

### EXAMPLES

### Analyses

The various analyses in the following examples were performed as follows.

The X-ray data were collected at room temperature on an Enraf-Nonius CAD-4 diffractometer with graphite-monochromated Moβα radiation, using an ω-2θ scan mode. ¹H, ¹³C, ¹¹B NMR spectra were run in a Varian Unity 300 MHz spectrometer; ¹H and ¹³C chemical shifts were referenced with the residual solvent resonances relative to tetra methyl silane. ¹¹B chemical shifts were referenced using an external solution of NaBH₄.

C, H, N analyses were performed on a EA110 CE Instruments automatic analyzer.

IR spectra were recorded as KBr pellets on a Perkin-Elmer 577 spectrometer.

FTICR/MS were obtained by electron impact (EI) with an Extrel (Waters) FTMS 2001-DT instrument.

### Example 1

### Production of a Chelating Agent of General Formula (I)

The synthesis of these chelating agents was performed by reacting borohydride or organoborohydride salts with adequate protic substrates, like mercaptoimidazoles, mercaptothiazolines or mercaptothiazoles, which may or may not contain a biomolecule (FIG. 1).

### 1a. Sodium trihydro(1-methyl-2-mercaptoimidazolyl)borate:Na[H₃B(tim^{Me})] (1)

To a stirred suspension of NaBH₄ (1.990 g, 34.70 mmol) in THF (100 mL) at 50° C. a solution of 2-mercapto-1-methyl imidazole (3 g, 26.3 mmol) in the same solvent (50 mL) was added dropwise. After complete addition, the mixture was stirred at 45° C. for 3 h and overnight at room temperature. The reaction mixture was filtered to remove any insoluble material. Purification of 1 was achieved by successive recrystallizations from THF/n-hexane. Yield: 34% (1.330 g, 8.9 mmol).

Analysis calculated for C₄H₈N₂SBNa: C, 32.03%; H, 5.38%; N, 18.68%. Found: C, 32.03%; H, 4.74%; N, 18.23%. IR Data (Nujol, υ/cm⁻¹): 2395, 2359 and 2279 (BH). ¹H NMR (300 MHz, CD₃CN, δ (ppm)): 3.43 (3H, s, CH₃—N), 6.61 (1H, d, J_{H-H}=2.1 Hz, CH), 6.64 (1H, d, J_{H-H}=2.1 Hz, CH). ¹³C NMR (75.4 MHz, CD₃CN, δ (ppm)): 34.9 (CH₃—N), 117.1 (CH), 124.0 (CH), 162.4 (C═S) ¹¹B NMR (96 MHz, CD₃CN, δ (ppm)): 22.3 (q, J_{B-H}=90.9 Hz).

### 1b. Lithium dihydromethyl(1-methyl-2-mercaptoimidazolyl)borate:Li[H₂B(Me)(tim^{Me})] (2)

To a stirred solution of Li(MeBH₃) (250 mg, 7.0 mmol) in 25 ml THF, kept at a temperature between −5 and 0° C., a THF solution of 2-mercapto-1-methyl imidazole, was added dropwise in a 1:1 molar ratio. After complete addition, the reaction mixture was stirred at low temperature, between −5 and 0° C., until all the Li(MeBH₃) has been consumed, as checked by ¹¹B NMR. Compound 2 was recovered as a white microcrystalline solid, by removal of the solvent under vacuum while keeping the solution at low temperature (0-5° C.).

¹H NMR (300 MHz, CD₃CN, δ (ppm)): −0.19 (3H, tr, J_{H-H}=5.7 Hz, CH₃—B), 3.43 (3H, s, CH₃—N), 6.63 (1H, d, J_{H-H}=2.1 Hz, CH), 6.70 (1H, d, J_{H-H}=2.1 Hz, CH). ¹¹B NMR (96 MHz, CD₃CN, δ (ppm)): 27.43 (tr, J_{B-H}=84.10 Hz).

### 1c. Sodium trihydro(2-mercaptothiazoline)borate:Na[H₃B(tiaz)] (3)

Compound 3 can be prepared as above described for 1, starting from sodium borohydride and 2-mercaptothiazoline (tiazH).

¹H NMR (300 MHz, CD₃CN, δ (ppm)): 2.97 (2H, d, J_{H-H}=8.4 Hz, CH₂), 3.91 (2H, d, J_{H-H}=2.1 Hz, CH₂). ¹¹B NMR (96 MHz, DMSO-d₆, δ (ppm)): 23.4 (q, J_{B-H}=91.9 Hz).

### 1d. Sodium trihydro(2-mercaptobenzothiazolyl)borate:Na[H₃B(Bzt)] (4)

Compound 4 was obtained almost quantitatively by treatment of a suspension of sodium borohydride in THF with an equimolar amount of 2-mercaptobenzothiazole, after 2h of reaction at room temperature.

¹H NMR (300 MHz, DMSO-d₆, δ (ppm)): 7.09 (1H, m, CH), 7.24 (1H, m, CH), 7.44 (1H, dd, CH), 7.56 (1H, d, CH). ¹³C NMR (75.37 MHz, DMSO-d₆, δ (ppm)): 117.5, 119.3, 122.3, 125.2, 130.2, 148.8, 188.3 (C═S). ¹¹B NMR (96 MHz, DMSO-d₆, δ (ppm)): 21.7 (q, J_{B-H}=100.7 Hz).

### 1e. Sodium trihydro(1-[4-((2-methoxyphenyl)-1-piperazinyl) butyl]-2-mercaptoimidazolyl)borate:Na[H₃B(tim^{Bu-Way})] (5)

Compound 5 was synthesized as described above for 1, starting from sodium borohydride and 1-[4-((2-methoxyphenyl)-1-piperazinyl)butyl]-2-mercaptoimidazole (tim^{Bu-WAY}H), containing a fragment of WAY-100635, which is a well known antagonist of 5-HT_{1A} receptors.

¹¹B NMR (96 MHz, DMSO-d₆, δ (ppm)): 22.8 (br). ¹H NMR (CD₃CN): δ 1.45 (2H, tr, J_{H-H}=7.5 Hz, CH₂), 1.67 (2H, tr, J_{H-H}=7.5 Hz, CH₂), 2.35 (2H, tr, J_{H-H}=7.5 Hz, CH₂—N), 2.49 (4H, br, CH₂—N, pip), 2.97 (4H, br, CH₂—N, pip), 3.78 (3H, s, CH₃—O), 3.92 (2H, tr, J_{H-H}=7.5 Hz, CH₂—N), 6.61 (1H, d, JH-H=2.1 Hz, CH), 6.65 (1H, br, CH), 6.86-6.97 (4H, m, Ph). ¹³C NMR (CD₃CN): δ 24.5, 28.0, 47.5, 51.4, 54.2, 55.8, 112.6, 116.1, 119.0, 121.8, 123.4, 124.2, 142.7, 153.3, 161.6. ESI-MS (CH₃CN) m/z: calcd for [C₁₈H₂₈N₄OSB]⁻ (found), 359.2 (359.3).

### Example 2

### Production of a Tricarbonyl Rhenium Complex with a Chelator of General Formula (I)

The synthesis of the complexes is readily achieved by reacting the chelators of general formula (I) with common Re(I) starting materials, such as (NEt₄)₂[ReBr₃(CO)₃]. In all the complexes the chelators act as monoanionic and tridentate ligands, which coordinate through the sulphur atom and two agostic hydrides, as confirmed by spectroscopic analysis (IR, ¹H and ¹¹B NMR) and by X-ray diffraction analysis for one of the complexes (FIG. 3).

### 2a. Tricarbonyl Rhenium Complex of trihydro(1-methyl-2-mercaptoimidazolyl)borate:[Re{κ³-H₃B(tim^{Me})}(CO)₃] (6)

Solid Na[H₃B(tim^{Me})] (30 mg, 0.20 mmol) was added to a suspension of (NEt₄)₂[ReBr₃(CO)₃] (100 mg, 0.13 mmol) in distilled water and the mixture was stirred for 2 h at room temperature. The mixture was then centrifuged to recover a pale yellow precipitate that was washed twice with 5 mL of distilled water. The solid was purified by silica-gel flash chromatography using CH₂Cl₂/n-hexane (50/50) as eluent. After removal of the solvent from the collected fractions, complex 6 was obtained as a microcrystalline yellow solid. Yield: 61.5% (32 mg, 0.08 mmol).

Analysis calculated for C₇H₈N₂SO₃BRe: C, 21.11%; H, 2.03%; N, 7.04%. Found: C, 21.69%; H, 1.82%; N, 6.87%. IR Data (KBr, ν/cm⁻¹): 2506 (w) (B═H), 1931 (vs) and 2043 (s) (C/O). ²H NMR (300 MHz, CDCl₃, δ (ppm)): −5.48 (2H, br, Re . . . H—B), 3.73 (3H, s, CH₃—N), 7.08 (1H, d, J_{H-H}=2.1 Hz, CH), 7.17 (1H, d, J_{H-H}=1.8 Hz, CH). ¹³C NMR (75.37 MHz, CDCl₃, δ (ppm): 34.8 (CH₃—N), 122.2 (CH), 123.2 (CH), 164.07 (C═S), 190.29 (CO), 193.49 (CO). ¹¹B NMR (96 MHz, CDCl₃, δ (ppm)): 53.27 (m)

### 2b. Tricarbonyl Rhenium Complex of dihydromethyl(1-methyl-2-mercaptoimidazolyl)borate:[Re{κ-MeH₂B(tim^{Me})}(CO)₃] (7)

0.1N Li[MeH₂B(tim^{Me})](125 mg, 0.833 mmol) was added to a solution of (NEt₄)₂-[ReBr₃(CO)₃] in NaOH. After stirring overnight at room temperature, an insoluble yellow solid was separated by centrifugation and dried. Compound 7 was purified by silica-gel flash chromatography using CH₂Cl₂/n-hexane (80/20) as eluent.

FTICR-MS-EI (+) (25° C., 12 eV): 412 (M⁺). IR Data (KBr, ν/cm⁻¹) 2035 (s) and 1929 (vs) (C/O). ²H NMR (300 MHz, CD₃CN, δ (ppm)): −5.52 (2H, br, B—H), 0.67 (3H, CH₃—B), 3.69 (3H, s, CH₃—N), 7.17 (1H, d, JH—H=2.1 Hz, CH), 7.45 (1H, d, J_{H-H}=2.1 Hz, CH). ¹³C NMR (75.37 MHz, CD₃CN, d (ppm)): 8.53 (CH₃—B), 35.11 (CH₃—N), 120.27 (CH), 125.56 (CH), 163.80 (C═S), 192.37 (CO), 195.10 (CO). ¹¹B NMR (96 MHz, CD₃CN, d (ppm)): 67.91 (tr, J_{B-H}=55 Hz).

### 2c. Tricarbonyl rhenium complex of trihydro(2-mercaptobenzothiazolyl)borate:[Re(CO)₃{K³—H₃B(bzt)}] (8)

Compound 8 was synthesized and purified as above described for 6, and was obtained in 55% isolated yield.

¹H NMR (CDCl₃) δ −5.67 (2H, br, J_{B-H}=88 Hz, B—H . . . Re), 6.60 (1H, br, B—H), 7.45 (1H, m, CH), 7.55 (1H, m, CH), 7.64 (1H, d, J_{H-H}=7.8 Hz, CH), 7.95 (1H, d, J_{H-H}=8.1 Hz, CH) ¹³C NMR (CDCl₃): δ 116.8, 122.9, 125.9, 127.7, 134.3, 144.0, 190.2, 192.3, 193.7. ¹¹B NMR (96 MHz, CD₃CN, δ (ppm)): 53.5 (m).

### 2d. Tricarbonyl rhenium complex of trihydro(1-[4-((2-methoxyphenyl)-1-piperazinyl)butyl]-2-mercaptoimidazolyl)borate:[Re{κ³-H₃B(tim^{Bu-Way})}(CO)₃] (9) (FIG. 2)

Compound 9 was prepared as described above for 6. Purification of 9 was done by silica-gel flash chromatography using a CH₂Cl₂/CH₃CN gradient, from 0% to 80% of CH₃CN.

¹H NMR (300 MHz, CDCl₃, δ (ppm)): −5.49 (2H, br, B—H), 1.56 (2H, m, CH₂), 1.88 (2H, m, CH₂), 2.47 (2H, tr, CH₂), 2.65 (4H, br, N—CH₂), 3.09 (2H, br, N—CH₂), 3.84 (O—CH₃), 4.10 (2H, tr, CH₂), 6.83-6.89 (4H, br, Ph), 7.09 (1H, d, J_{H-H}=2.1 Hz, CH), 7.20 (1H, d, J_{H-N}=2.1 Hz, CH). ¹³C NMR (75.37 MHz, CDCl₃, δ (ppm)): 23.6, 26.9, 48.1, 50.5, 53.4, 55.3, 57.7, 111.0, 118.1, 121.0, 122.1, 122.4, 123.0, 141.0, 152.2, 163.80 (C═S), 193.6 (CO). ¹¹B NMR (96 MHz, CD₃CN, δ (ppm)): 52.9 (br).

### Example 3

### Production of a Tricarbonyl ^{99m}Tc Complex with a Chelator of General Formula (I): [^{99m}Tc (κ³-H₃B(Tim^{Me}))(CO)₃] (6a) (FIG. 4)

In a nitrogen-purged glass vial, 100 μL of a 2.5×10⁻² M aqueous solution of ligand 1 were added to 400 μL (7.5 mCi) of the organometallic precursor fac−[^{99m}Tc(OH₂)₃(CO)₃]⁺, and the mixture was incubated at room temperature for 30 min. After this time, complex 6a has been obtained in 90% yield, as checked by gradient HPLC analysis (from 100% aqueous 0.1% CF₃COOH solution to 100% CH₃CN) using a Nucleosil column (10:m, 250 mm×4 mm). The chemical identity of 6a was confirmed by comparing its HPLC chromatogram with the HPLC profile of the analogue Re complex (6) (Retention time: Re, 25.78 min; ^{99m}Tc, 26.28 min). HPLC analysis of the preparation of 6a, at different intervals of time, has shown that 6a is stable under physiologic conditions (PBS, pH 7.4), at least for a period of 24 h.

1. Compound, suitable for use as a chelator in the radioactive labeling of biomolecules with metal tricarbonyl complexes, which compound has the general formula I 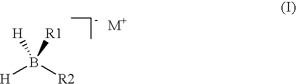
wherein
M is a monovalent cation, such as Li, Na, K, Tl, Rb, Cs or an alkyl ammonium;
R1 is H, alkyl, aryl or a biomolecule;
R2 is H or a pendant arm, said pendant arm optionally comprising a biomolecule,
with the proviso that when R1 is H, R2 is not H or COOH, and when R1 is alkyl or aryl, R2 is not H.

2. Compound as claimed in claim 1, wherein the pendant arm is a coordinating group.

3. Compound as claimed in claim 2, wherein the coordinating group is selected from mercaptoimidazoles, mercaptothiazoles, mercapto-oxazoles, mercaptoimidazolines mercaptothiazolines, mercapto-oxazolines, mercaptobenzothiazoles, mercaptobenzimidazoles, mercaptobenzoxazoles, pyrroles, furans, thiophenes, imidazoles, oxazoles, thiazoles, pyrazoles, pyridines and pyrimidines or groups containing thioethers, amines or carboxylates.

4. Compound as claimed in claim 1, wherein the pendant arm is a non-coordinating group.

5. Compound as claimed in claim 4, wherein the non-coordinating group is alkyl or aryl.

6. Compound as claimed in claim 5, wherein the alkyl is selected from methyl, ethyl, n-propyl, isopropyl, n-butyl, isobutyl, s-butyl, t-butyl, n-pentyl, isopentyl, neopentyl, hexyl, 2-methylpentyl, 3-methylpentyl, 2,3-dimethylbutyl, or 2,2-dimethylbutyl.

7. Compound as claimed in claim 1, wherein the aryl is monocyclic or polycyclic, which polycyclic aryls optionally have one or more rings that are not aromatic, and said aryls are further optionally substituted with an alkyl group.

8. Compounds as claimed in claim 1, wherein the alkyls and aryls have functional groups selected from carboxyl (COOH), amines (NHR, wherein R is H, alkyl or aryl), aldehydes ((C═O)H), hydroxy (OH), mercapto (SH) or ester (COOR, wherein R is alkyl or aryl) for coupling biomolecules.

9. Compounds as claimed in claim 1, wherein the aryl is phenyl that is optionally substituted with methyl, ethyl, isopropyl or n-propyl.

10. Compound as claimed in claim 1, having a combination of R1 and R2 as listed in Table 1.

11. Compound as claimed in claim 1, wherein the biomolecule is selected from CNS receptor ligands, steroids, sugars, amino acids, substrates or inhibitors of enzymes, or molecules with affinity for receptors over-expressed in certain tumor cells.

12. Compound as claimed in claim 11, wherein the CNS receptor ligand is selected from antagonists of dopamine and serotonin transporters, dopamine and serotonin receptor ligands or small molecules for targeting β-amyloid plaques.

13. Compound as claimed in claim 11, wherein the steroid is selected from estrogens, progestens, androgens or derivatives thereof.

14. Compound as claimed in claim 11, wherein the sugar is selected from glucose, mannose or derivatives thereof.

15. Compound as claimed in claim 11, wherein the molecule with affinity for receptors over-expressed in certain tumor cells is selected from peptides and from sigma receptor ligands.

16. Compounds as claimed in claim 11, wherein the amino acids are selected from methionine, phenylalanine or tyrosine.

17. Compounds as claimed in claim 11, wherein the substrates or inhibitors of enzymes are selected from L-arginine or quinazoline derivatives.

18. Method for the preparation of a compound as claimed in claim 1, comprising reacting a compound of the general formula H₃BR1, wherein R1 is H, alkyl, aryl or a biomolecule, with a compound selected from mercaptoimidazoles, mercaptobenzothiazoles, mercaptothiazoles, mercapto-oxazoles, mercaptoimidazolines, mercaptothiazolines, mercapto-oxazolines or any one of these compounds linked to a biomolecule.

19. Compound of the general formula: 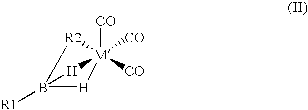
wherein M′ is a transition metal, such as a group VII element, and R1 and R2 are as defined in claim 1, wherein the proviso to claim 1 does not apply.

20. Compound as claimed in claim 19, wherein M′ is rhenium, technetium or manganese, more in particular a radioactive isotope of rhenium, technetium or manganese.

21. The compound as claimed in claim 20, wherein M′ is one of ^{99m}Tc and ^{186/188}Re.

22. Compound as claimed in claim 19, wherein R1 is or is substituted with a biomolecule.

23. Compound as claimed in claim 19, wherein R2 is or is substituted with a biomolecule.

24. Compound as claimed in claim 19, wherein R1 is a biomolecule and R2 is substituted with a biomolecule.

25. Method for the preparation of a compound as claimed in claim 19, comprising contacting a metal tricarbonyl with a compound as claimed in claim 1, wherein the proviso to claim 1 does not apply.

26. Compound as claimed in claim 22, for use in therapy or diagnosis.

27. Compound as claimed in claim 22, for use in diagnosis and/or therapy of cancer or other hyperproliferative and/or neoplasic conditions.

28. Use of a compound as claimed in claim 22, for the preparation of a pharmaceutical composition for the treatment and/or diagnosis of cancer or other hyperproliferative and/or neoplasic conditions.

29. Use of a compound of the general formula I, 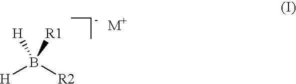
wherein M, R1 and R2 are as defined in claim 1,
as a chelator in the radioactive labeling of biomolecules with metal tricarbonyl complexes,
wherein the proviso to claim 1 does not apply.
